(19) United States
(12) Patent Application Publication
(10) Pub. No.: US 2018/0028398 A1
(43) Pub. Date: Feb. 1, 2018

(54) Erectile Dysfunction Device

(71) Applicant: Kenneth La'Shawn Ford, SR., Salem, OR (US)

(72) Inventor: Kenneth La'Shawn Ford, SR., Salem, OR (US)

(21) Appl. No.: 15/221,310

(22) Filed: Jul. 27, 2016

(51) Int. Cl. A61H 19/00; A61F 5/41

(52) U.S. Cl. CPC A61H 19/44; A61H 19/34; A61H 19/32; A61H 19/50; A61F 5/41

(57) ABSTRACT

A reusable, cloak wearable over the male member, capable of enhancing or maintaining the blood flow that helps establish and maintain an erection. It has a linear, slideable tubular sleeve that resides atop of a base sheath that encapsulates the penile shaft and glans penis. The sheath has a washable inner, compressible flesh-like lining that adds girth and conforms to the dimensions of the user. The tip of the glans fits securely into the distal end of an extending tip. During coitus, the linear motion of the tubular sleeve coupled with its physical surface configuration adds additional stimulation to drive additional blood into the penis. The cloak is held in place by a testicular lasso that encircles the back side of the scrotal sac to aid in constraining the blood engorgement of the male member's two corpora cavernosa and single corpus spongiosum.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to a personal device for the enhancement of both partner's sexual pleasure during coitus, as well as for the treatment of erectile dysfunction.

### BACKGROUND

Studies show that 5% of 40-year-old men and between 15% and 25% of American men over the age of 65 have some level of erectile dysfunction, with numerous others have trouble establishing and maintaining an erection. While there are a plethora of medications that treat this problem, there are inherent side effects such as vision problems, dizziness, body aches and chest pain. Further, a percentage of users report issues in returning to a flaccid state—a condition that requires immediate medical attention. Lastly, many men cannot combine these medications with other medications they are presently taking while others are adverse to the use of elective medications.

From the female perspective, many women fail to reach climax during coitus unless their male partner has a certain girth or length. men without the ability to bring their partner to climax because of their dimensions often feel inadequate or see themselves as poor lovers. these problems with erectile dysfunction or incompatible dimensions have caused countless marital infidelities and or marital dissolutions.

There is a long felt need in for a device that will held men reach and maintain an erection as well as for a device that will increase the component of sexual pleasure for both partners. The present invention provides a reusable, wearable mechanical device that provides a solution to all the aforementioned problems.

### BRIEF SUMMARY

In accordance with various embodiments, a wearable mechanical device that is customizable to allow for additional stimulation of the male and female sex organs is provided.

It is another aspect of the present invention to provide a device that will aid in the establishment and maintenance of an erection.

It is a further aspect of the present invention to provide a reusable, washable girth and length enhancer for the male member that may be specifically modified to suit one's particular situation.

It is yet another aspect of the present invention to provide a securely attachable male member enhancer, conformable to the individual wearer and that is unlikely to loosen during coitus.

It is a final aspect of the present invention to provide a personal device that targets solving erectile dysfunction and enhancing the pleasure of coitus so as to aid in the maintenance of relationship fidelities and marital bliss.

### BRIEF DESCRIPTION OF THE DRAWINGS

A further understanding of the nature and advantages of particular embodiments may be realized by reference to the remaining portions of the specification and the drawings, in which like reference numerals are used to refer to similar components.

FIG. 1 is a side view of a first embodiment erectile dysfunction device worn by a man;

FIG. 2 is a side view of a first stimulation slider removed from the shaft portion of the first embodiment erectile dysfunction device;

FIG. 3 is a cross sectional view of a first embodiment erectile dysfunction device worn by a man;

FIG. 4 is a representative view of a coital penetration stroke with a first embodiment erectile dysfunction device worn by a man;

FIG. 5 is a representative view of a coital withdrawal stroke with a first embodiment erectile dysfunction device worn by a man;

FIG. 6 is a partial side view of a second stimulation slider installed on the shaft portion of the first embodiment erectile dysfunction device;

FIG. 7 is a rear perspective view of the second embodiment erectile dysfunction device;

FIG. 8 is a side view of the second embodiment erectile dysfunction device;

FIG. 9 is a side view of the second embodiment erectile dysfunction device without additional stimulation rings;

FIG. 10 is a side view of the second embodiment erectile dysfunction device with additional stimulation rings;

FIG. 11 is a side cross sectional view of the second embodiment erectile dysfunction device,

FIGS. 12-14 are front perspective views of the second embodiment erectile dysfunction device's various stimulation rings;

FIG. 15 is a side view of the third embodiment erectile dysfunction device;

FIG. 16 is a side perspective view of the third embodiment erectile dysfunction device; and

FIG. 17 is a side view of the fourth embodiment erectile dysfunction device worn by a man.

### DETAILED DESCRIPTION OF CERTAIN EMBODIMENTS

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers herein used to express quantities, dimensions, and so forth, should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

The term “EDD” as used herein refers to any of the various embodiments of the erectile dysfunction device of the present invention as disclosed herein.

The term “penile sock” as used herein refers to a sheath for the encasement of a male penis having a closed distal end and an open proximal end that allows for the entry of a penis. It may have other openings along its length.

The term “testicular lasso” and “scrotal lasso” are interchangeable terms as used herein that refer to an elastic loop affixed to the EDD, that when stretched can encircle the base of the scrotum and maintain the position of the EDD on the length of the penis.

The term “erectile dysfunction” as used herein is the inability to get or keep an erection firm enough to have satisfactory sexual intercourse. It can describe a complete or inconsistent inability to achieve an erection, or a tendency to sustain only brief or weak erections.

The term “stimulation slider” is a hollow sleeve adapted for linear and/or rotational movement bout a penile sock

The present invention relates to a novel design for an erectile dysfunction device.

An erection results from changes in blood flow in the penis. When a man becomes sexually aroused, nerves cause the two main penis arteries to expand and at the same time, for the penis veins to close up. More blood flows in and less flows out of the penis. The blood fills and hardens the two columns of tissue running along the sides of the penis known (the corpus cavernosum). Once blood is in the penis, pressure traps it within the corpora cavernosa (the tunica albuginea acts like a “trap door”) keeping the blood in making the penis expand and maintaining the erection.

Achieving and maintain an erection is a complex event. Erectile dysfunction can be the result of several factors such as aging, lifestyle, medication, vascular disorders, psychological disorders, neurological disease, endocrine abnormalities and chronic illness to name a few. The erectile dysfunction device (EDD) aids in both the achievement of an erection through sensory enhancement and psychological stimulation. It also helps maintain an erection by providing additional stimulation during coitus and by adding an additional “trap door” to slow or stop the leakage of blood from the corpus cavernosum. (This is important for men with vascular disorders, or endocrine abnormalities.) Lastly, the added girth of the EDD increases the pressure or compressive forces that the penis experiences while in the vagina, increasing the stimulation. (This is important for men with decreased sensation in their penis.)

A secondary function of the EDD inures to the female partner. It is designed to add additional clitoral and vaginal stimulation during coitus. It does this with the added girth and length of the penile sock an the shape and motion of the stimulation sleeve 14. By increasing sexual arousal in the female partner, the psychological effects the male partner experiences seeing his partner rise to new levels of stimulation increases his own psychosexual arousal level, aiding in the maintenance of his erection.

The present invention may be used in conjunction with other erectile dysfunction treatment options, such as psychotherapy, drug therapy, oral medications, injectable medications, vacuum devices, and surgery.

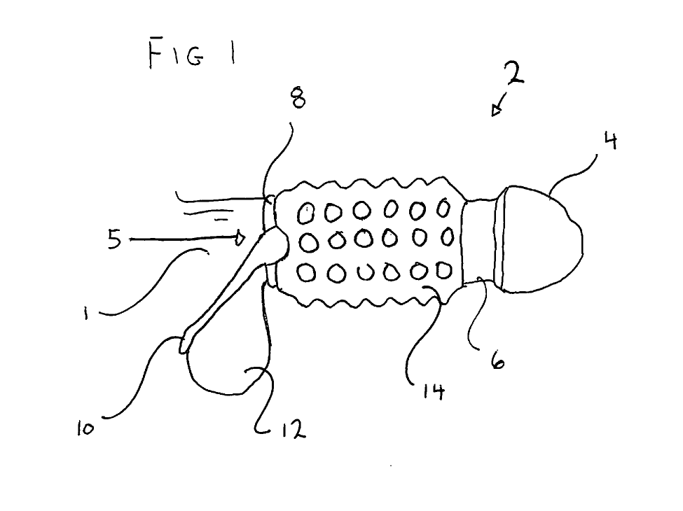

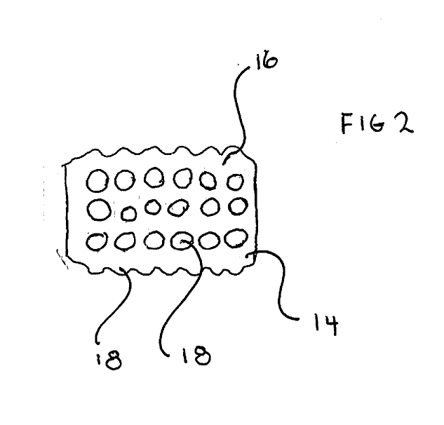

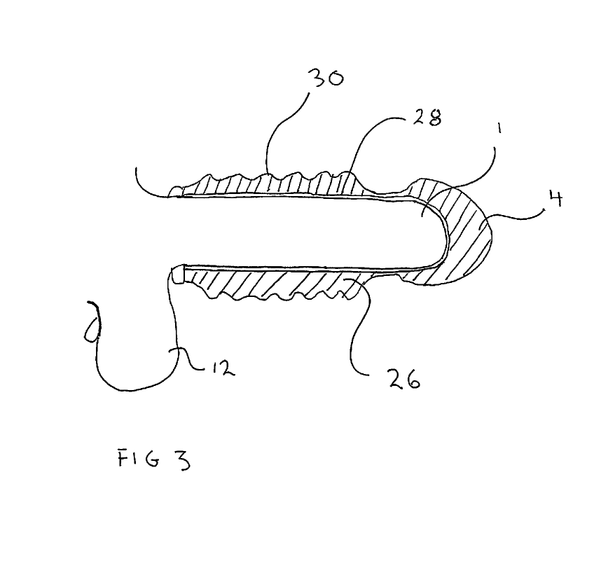

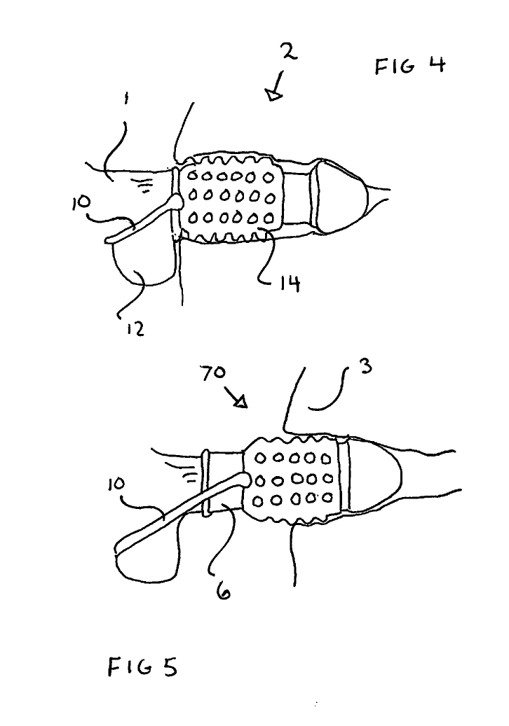

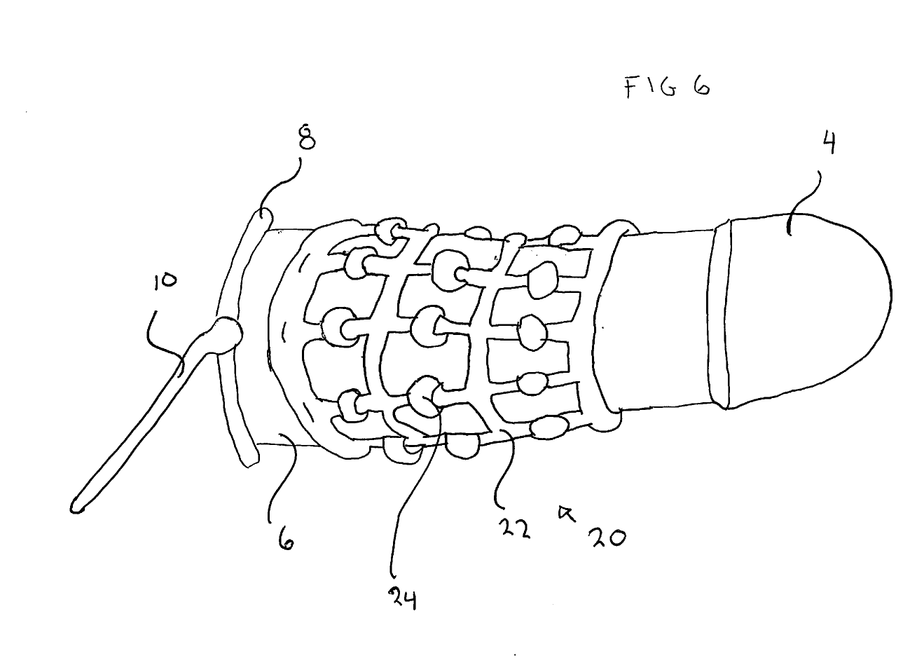

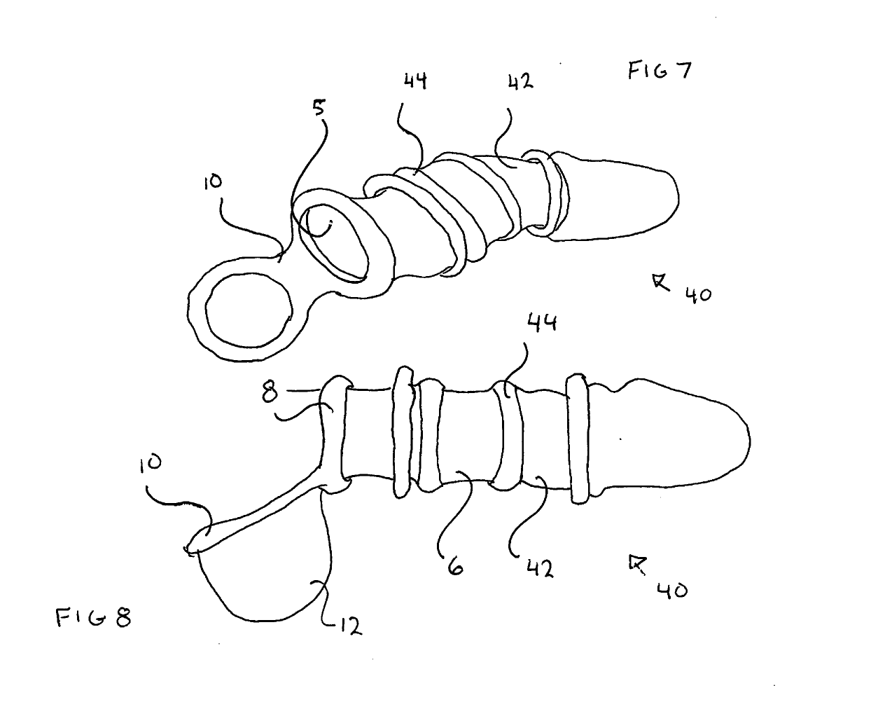

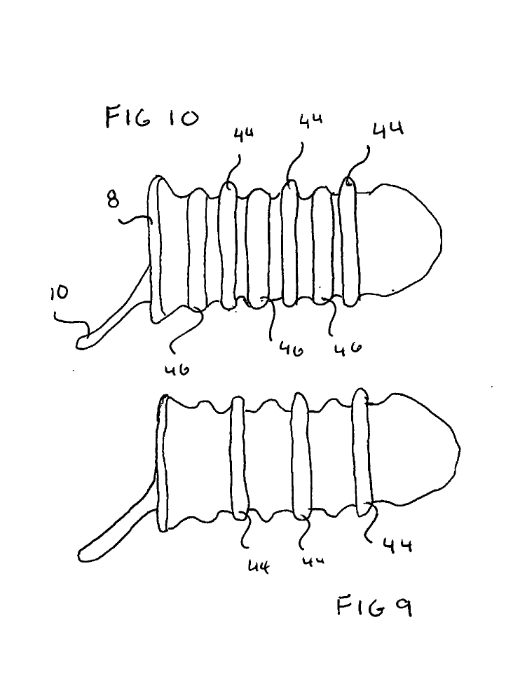

Looking at FIGS. 1 and 4 it can be seen that the first and preferred embodiment EDD 2 is a penile sock adapted to be worn completely over the head and shaft of the male penis 1 coupled with an interchangeable surface embossed stimulation slider 14. The distal end of the sock has a bulbous head 4 formed thereon. The middle section is a smooth tubular shaft 6. The proximal end of the sock has a circular stop flange 8 extending normally from and formed at the end of the shaft 6 (or at the proximal end of the EDD) adjacent the opening 5. Affixed to the stop flange 8 and the tubular shaft 6 is an elastic testicular lasso 10 adapted for firm, stretched engagement about the male scrotal sac 12 behind the testes (not pictured). Slidingly housed on the tubular shaft 6 between the stop flange 8 and the base of the head 4 is a hollow, cylindrical, open ended, tube or stimulation slider 14. The slider is capable of bidirectional linear motion between the head 4 and the stop flange 8, along the outside surface of the shaft 6. The difference in the outer diameter size of the uncompressed head 4 and the diameter of the inside of the hollow stimulation slider 14 prevents the slider 14 from coming off of the distal end of the EDD. Similarly, the difference in the uncompressed outer diameter size of the stop flange 8 and the diameter of the inside of the hollow stimulation slider 14 prevents the slider 14 from coming off of the proximal end of the EDD.

It is to be noted that in a modification of this embodiment as shown in FIG. 5 the elastic testicular lasso 10 adapted for firm, stretched engagement about the male scrotal sac 12 behind the testes is not coupled to the stop flange 8 but rather is affixed to the proximal end of the stimulation slider 14. In this manner during the withdrawal stroke of coitus the distance between the stimulation slider 14 and the scrotal sac is increased, thereby increasing the grip of the testicular lasso 10 about the scrotal sac. With the alternating penetration and withdrawal strokes, this modified embodiment acts to pump more blood into the penis and temporarily increase the localized blood pressure, thereby increasing stimulation to the male.

Looking at FIG. 2, the preferred embodiment stimulation slider 14 is a hollow, open-ended tube sized for sliding engagement over the penile sock between the head 4 and the stop flange 8. Its inner surface is preferably smooth to allow for ease of linear movement along the shaft 6 although in other embodiments a surface configuration may be employed to reduce the amount of movement and to transmit point stimulation to the underlying male penis. The outer surface 16 is adorned with an array or series of raised bumps 18 to increase the female partner's vaginal and clitoral stimulation as well as increase the compression forces of the vagina about the penis. It cannot be removed off of the penile sock while it the penile sock is worn.

Removal for replacement, cleaning or lubrication must be done when the EDD 2 is not worn. Removal of the stimulation sleeve 14 is accomplished by compression of the proximal end of the penile sock so as to deform or “scrunch” the proximal end of the sock, the stop flange 8 and the testicular lasso 10 into a configuration with a small enough profile to allow the inside of the stimulation sleeve 14 to pass over top of them and off the penile sock.

The stimulation sleeve 14 may take many different configurations. A beaded stimulation sleeve 20 can be seen in FIG. 6. Here it retains the hollow open-ended tubular shape as in the preferred embodiment but the tube section does not have a continuous wall but rather its tube shape is formed from a netting 22 with a series of beads 24 rotatably mounted on the netting.

Looking at FIG. 3, the wearing, and design of the EDD 2 can best be explained. Here it can be seen in cross section, that the male penis 1 occupies much less than the full volume of the penile sock. The function of the EDD 2 is to also add girth and length to the male member for the purposes detailed herein. The male member 1 is held in the penile sock by the frictional fitting between the penis 1 and the inner face 28. The inner face 28 is firmly forced onto the outside of the penis 1 by compression of the liner 26 which is a concave, tubular, compressible, elastically deformable sleeve. (A washable foam is preferable.). The proximal end of the penile sock is open for insertion of the male member and the distal end is closed, allowing support to the entire glans 32. The liner 26 extends along the inner length of the shaft of the EDD and encompasses part of the inner distal tip of the bulbous head 4. This is how the EDD 2 adds girth and length to the male member.

The materials of construction of the penile sock and the stimulation slider are of a material that mimics the experience of human skin; warm and soft to the touch, yet with a firm underlying texture. It has many different materials that are used ranging from elastomers, (material without phthalates), silicon, rubber latex, thermoplastic rubber, “jelly” (a mixture of PVC and rubber), and other products imparting the desired valued of human-like feel.

The inside liner 26 is made of an elastically deformable material adapted to conform to the outline of the male user's penis 1 and snugly cradle its entirety. Available fabricated from numerous open, closed or semi open cell polymers, such a material is commonly referred to in the industry as “memory foam.” The liner 26 is held between the secondary layer inner face 28 and the first layer outer face 30 of the penile sock.

FIGS. 4 and 5 illustrate the operation of the first embodiment EDD 2 and modified first embodiment EDD 70 during coitus as the operation of the stimulation slider 14 is identical. On the coital penetration stroke of FIG. 4 as the penis 1 further penetrates the vagina 3, the stimulation sleeve 14 remains in moving contact with the clitoris sliding back along the shaft 6 of the penile sock until it abuts the stop flange 8 and then remains at the proximal end of the penis 1 but fully inserted into the vagina 3. The penis 1 is cradled snuggly inside the penile sock by flesh-like material which transmits the pressure of the vaginal walls to the underlying penis 1. The testicular lasso 10 surrounds the base of the scrotal sac constraining the blood that maintains the erection of the penis 1. On the coital withdrawal stroke of FIG. 5 the penis 1 is partially removed from the vagina 3 with the testicular lasso 10 additionally ensuring that the penis 1 does not exit the penile sock. Since the stimulation sleeve 14 is not directly affixed to the penile sock, and because the stimulation sleeve 14 is in frictional contact with the vagina 3, it is free to slide in a linear fashion along the shaft 6 toward the head 4 of the EDD. The result is that the stimulation sleeve 14 withdraws from the vagina 3 at a slower rate than the underlying male member 1, massaging the shaft of the penis 1 and remaining in contact with the clitoris at all times. (Allowing its outside surface of raised bumps to act to continually massage the clitoris.) This motion of the thin stimulation sleeve 14 is also transmitted vibrationally through the penile sock and liner 26 to the underlying penis 1. (It is to be noted here that the stimulation sleeve 14 is free to rotate about the shaft 6 as well as traverse along its length. This comfortably accommodates change in sexual positions.) As discussed above, the testicular lasso of modified version of the first embodiment EDD 70 acts to tighten on the withdrawal stroke of coitus so as to add blood and increase the localized blood pressure in the penis.

Use of the EDD as disclosed herein adds girth and length (for increased female physical stimulation and increased male psyco-sexual stimulation), adds additional pressure about the male member (for increased male stimulation). retains additional blood in the penis (to maintain erection), increases clitoral and vaginal stimulation (for increased female physical stimulation and increased male psyco-sexual stimulation), adds additional stroke and vibrational stimulation along the shaft of the penis (for increased male stimulation). Thus, many of the factors attributing to erectile dysfunction are addressed with use of the EDD.

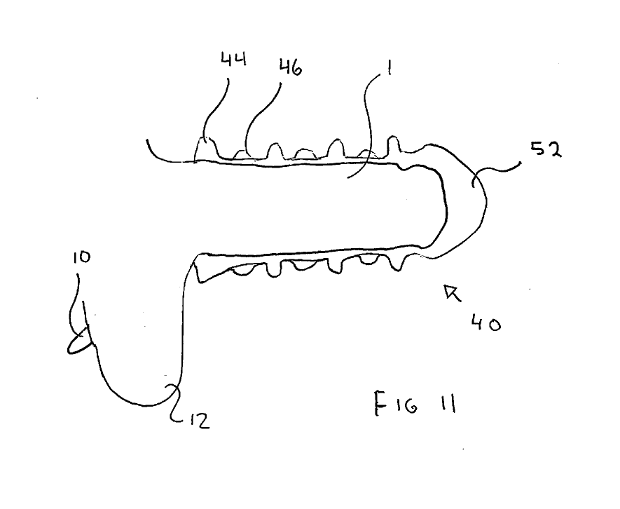

Looking at FIGS. 7-13 the second embodiment EDD 40 can best be described. The second embodiment EDD 40 has a modified penile sock 42 also having a bulbous head 4 at the distal end and an opening 5 at the proximal end with an intermediary shaft 6. There is also a testicular lasso 10 affixed at the proximal end of the penile sock or the stop flange 8. The middle section has a smooth tubular shaft 6 with a series of circumferential retention rings 44 rigidly affixed at spaced intervals along the length of the shaft 6, and extending normally therefrom.

Between the fixed retention rings 44 may be placed floating stimulation rings 46 having circular orifices therethrough. These are placed there in the same way that the stimulation sleeve 14 is placed on the first embodiment EDD 2 described above. The inside diameter of the orifice through the stimulation ring 46 is larger than the outside diameter of the shaft but smaller than the outside diameter of the retention rings 44. They are free for rotational and sliding linear motion between the adjacent fixed retention rings 44. These function during coitus in the same way that the stimulation sleeve 14 functioned on the first embodiment EDD 2. In the preferred embodiment these stimulation rings 46 may vary in durometer from rigid to flexible.

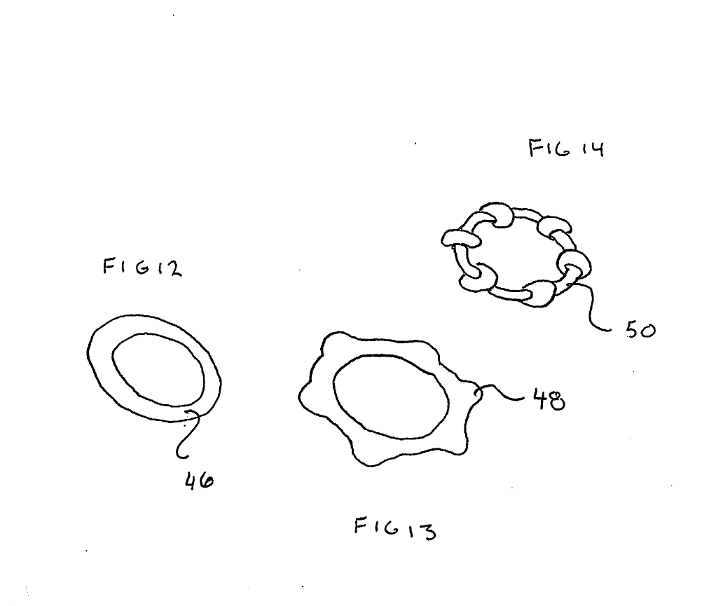

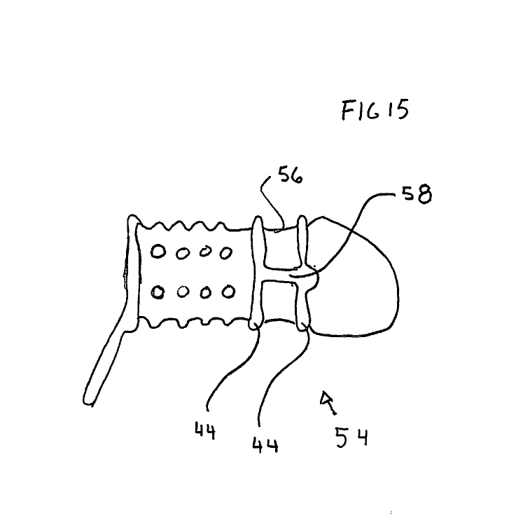

The floating stimulation rings 46 vary in design and configuration as well as the density and materials of construction. FIG. 12 shows a simple, unadorned floating stimulation ring 46. FIG. 13 shows a raised ridge stimulation ring 48 having a non uniform outer diameter, and FIG. 14 shows a beaded roller floating stimulation ring. The operation of this second embodiment EDD 40 is substantially similar to that of the preferred embodiment with the primary difference being that there is no secondary layer inner face since there is no elastically deformable sleeve. However, as FIG. 11 illustrates, there is an elastically deformable, compressible extender tip built into the head 4. So while length is added in the second embodiment, there is no overall girth added to the male member. From an erectile dysfunction perspective, this second embodiment EDD 40 addresses many of the same factors as the preferred embodiment in the same way.

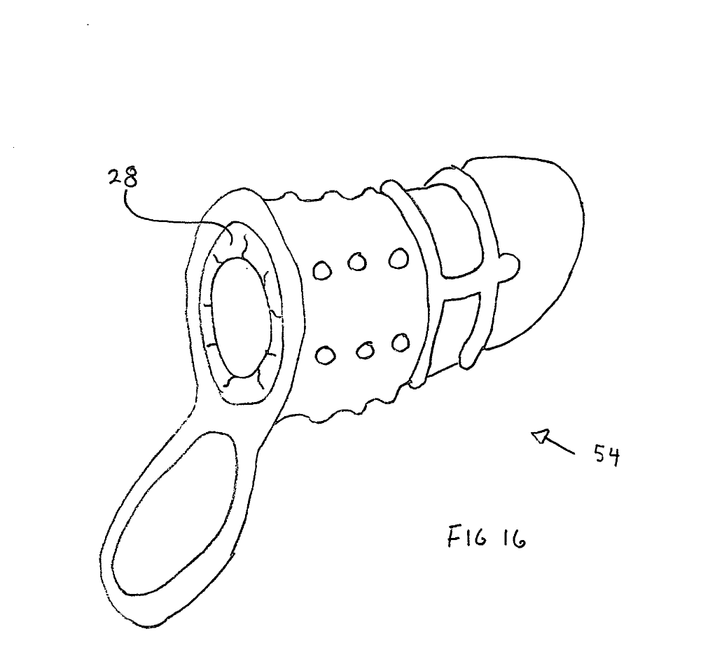

The third embodiment EDD 54 is seen in FIGS. 15 and 16. This varies from the first embodiment EDD 2 only because it incorporates an upper, open region 56 on the shaft with only a very thin first layer outer face 30 on the penile sock in that region. There is a pair of fixed retention rings 44 on either side of the open region 56 connected by at least one elastic, flexible rib 58. Besides adding increased vaginal wall and clitoral stimulation, these retention rings 44 and their ribs 58 serve to ensure the third embodiment EDD 54 does not tear apart where it is only connected with the very thin first layer outer face 30. The rear perspective view of FIG. 16 illustrates the secondary layer inner face as it contains the elastically deformable sleeve. The stimulation slider may be permanently affixed to the proximal end of the penile sock or it may be unconnected, allowing for limited linear movement along the penile sock.

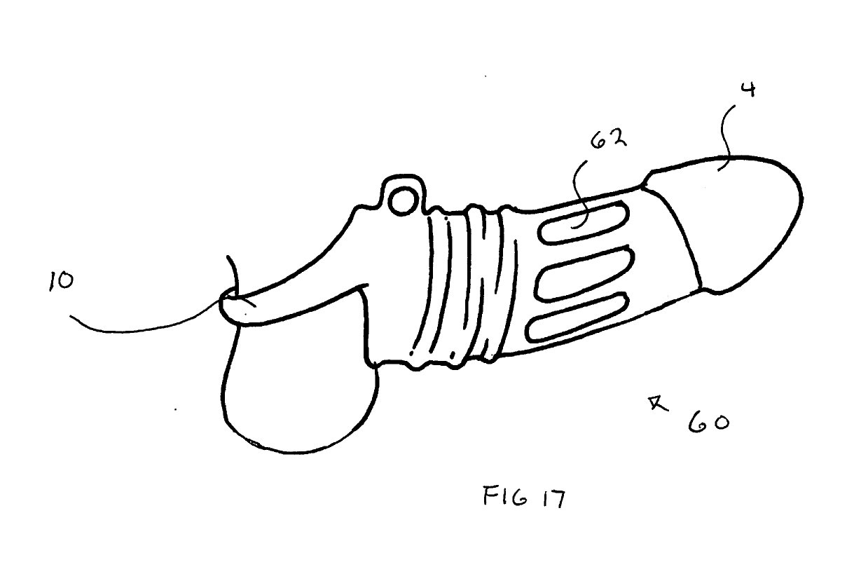

FIG. 17 illustrates the fourth embodiment EDD 60. This version is a condom with open slots 62 along its length, an affixed testicular lasso 10 and a compressible extender tip built into the head 4 similar to that of the second embodiment EDD 40, Affixed around the proximal base of the EDD 60 is a battery clitoral excitation vibrational ring as is well known in the art of sex toys. Here this serves for erectile dysfunction purposes, namely the retention of additional blood in the penis (to maintain erection), increased clitoral and vaginal stimulation (for increased female physical stimulation and increased male psyco-sexual stimulation), and additional vibrational stimulation at the base of the penis (for increased male stimulation).

While certain features and aspects have been described with respect to exemplary embodiments, one skilled in the art will recognize that numerous modifications are possible. While certain functionality is ascribed to certain system components, unless the context dictates otherwise, this functionality can be distributed among various other system components in accordance with the several embodiments.

System components described according to a particular structural architecture and/or with respect to one system may be organized in alternative structural architectures and/or incorporated within other described systems. Hence, while various embodiments are described with—or without—certain features for ease of description and to illustrate exemplary aspects of those embodiments, the various components and/or features described herein with respect to a particular embodiment can be substituted, added, and/or subtracted from among other described embodiments, unless the context dictates otherwise. Consequently, although several exemplary embodiments are described above, it will be appreciated that the invention is intended to cover all modifications and equivalents within the scope of the following claims.

Having thus described the invention, what is claimed as new and desired to be secured by Letters Patent is as follows:

1. An erectile dysfunction device comprising:
a penile sock having a closed distal end and an open proximal end;
a removable, stimulation slider mounted about said penile sock for sliding linear motion;
an elastic testicular lasso affixed to said proximal end of said penile sock;
and wherein said stimulation slider is a hollow cylindrical sleeve.

2. The erectile dysfunction device of claim 1 further comprising:
a stop flange extending normally from said proximal end of said penile sock; wherein said stop flange has an outer diameter that exceeds an inner diameter of said stimulation slider.

3. The erectile dysfunction device of claim 2 wherein said penile sock is a tubular shaft with a bulbous head formed at said closed distal end;
wherein said bulbous head has an outer diameter larger than said inner diameter of said stimulation slider.

4. The erectile dysfunction device of claim 3 further comprising:
an elastically moldable and compressible inside liner;
and wherein said penile sock has a first layer outer face and a second layer inner face wherein said inside liner is disposed between said outer face and said inner face, extending into an interior void of said bulbous head so as to fill the distal tip of said head.

5. The erectile dysfunction device of claim 1 wherein said stimulation slider is surface embossed with an array of raised bumps.

6. The erectile dysfunction device of claim 1 wherein said stimulation slider is a netting tube with an array of rotatable beads affixed thereto.

7. The erectile dysfunction device of claim 1 wherein said testicular lasso is affixed to said stop flange of said penile sock.

8. An erectile dysfunction device comprising:
a penile sock having a bulbous head at a distal end, an opening at a proximal end, and a intermediary shaft with a first external diameter, said shaft residing there between said head and said opening;
an elastic testicular lasso affixed to said proximal end of said penile sock;
at least two circumferential retention rings extending normally from and rigidly affixed at spaced intervals along a length of said shaft, said retention rings having a second external diameter.

9. The erectile dysfunction device of claim 8 further comprising:
at least one stimulation ring having an internal circular orifice formed there through, said circular orifice having an internal diameter, and wherein said stimulation ring resides between adjacent fixed retention rings about said shaft and capable of linear and rotational movement between said adjacent retention rings.

10. The erectile dysfunction device of claim 9 wherein said internal diameter of said stimulation ring exceeds said first outer diameter of said shaft but is less than said second outer diameter of said retention ring.

11. The erectile dysfunction device of claim 10 further comprising:
a extender tip built into the internal, distal end of said bulbous head, said tip made of an elastically deformable, compressible material.

12. The erectile dysfunction device of claim 11 wherein said stimulation ring has a non uniform outer diameter.

13. An erectile dysfunction device comprising:
a penile sock having a closed distal end and an open proximal end;
a hollow sleeve stimulation slider mounted about said proximal end of said penile sock;
a stop flange extending normally from and attached to said proximal end of said penile sock;
an elastic testicular lasso affixed to said proximal end of said penile sock;
a first retention ring affixed to and extending normally from said penile sock, residing between said proximal end and said distal end of said penile sock;
a bulbous head with a second retention ring affixed to and extending normally from a proximal end of said bulbous head, and
at least one rib connecting said first retention ring and said second retention ring; and
wherein said first retention ring and said second retention ring reside in a spaced configuration from each other

14. The erectile dysfunction device of claim 13 wherein said stimulation slider is surface embossed with an array of raised bumps.
